(19) United States
(12) Patent Application Publication
(10) Pub. No.: US 2023/0166011 A1
(43) Pub. Date: Jun. 1, 2023

(54) ISCHEMIC HEART DISEASE ANIMAL MODEL USING 3-DIMENSIONAL BIOPRINTED OCCLUDE, AND MANUFACTURING METHOD THEREFOR

(71) Applicants: POSTECH Research and Business Development Foundation, Pohang-si (KR); INDUSTRY FOUNDATION OF CHONNAM NATIONAL UNIVERSITY, Gwangju (KR)

(72) Inventors: Jinah JANG, Pohang-si (KR); Han Byul KIM, Gwangju (KR); Young Joon HONG, Gwangju (KR); Seungman JUNG, Gimpo-si (KR)

(21) Appl. No.: 17/912,480

(22) PCT Filed: Jul. 7, 2020
(86) PCT No.: PCT/KR2020/008827

§ 371 Date: Sep. 16, 2022

(30) Foreign Application Priority Data
Mar. 20, 2020 (KR) 10-2020-0034704

(51) Int. Cl. A61L 31/14; A61L 31/04; B29C 64/10; B33Y 10/00; B33Y 70/00; B33Y 80/00

(52) U.S. Cl. CPC A61L 31/14; A61L 31/04; B29C 64/10; B33Y 10/00; B33Y 70/00; B33Y 80/00

(57) ABSTRACT

The present invention relates to an animal model with ischemic heart disease, by a 3-dimensional bioprinted occluder and method for producing the same.

### TECHNICAL FIELD

The present invention relates to a hollow construct prepared by a 3-dimensional bioprinting method for preparing an ischemic heart disease animal model and an ischemic heart disease animal model using thereof, and more specifically, it relates to a hollow construct for preparing a myocardial infarction model manufactured by 3-dimensional bioprinting technology, an animal model with myocardial infarction using thereof, and a method for preparation of an animal model, and relates to a large animal myocardial infarction model necessary for a non-clinical experiment.

### BACKGROUND ART

Myocardial infarction is a disease with the highest incidence and mortality worldwide, and various research and development are required to treat it. When myocardial infarction occurs, the coronary artery in the heart structure is narrowed, which prevents the myocardium from receiving sufficient oxygen and nutrients. As a result, the myocardium near the coronary artery reaches a state of necrosis, resulting in failure to perform a normal function.

The necessity for animal experiments to treat myocardial infarction was continuously required, and by developing myocardial infarction models in rodents, sheep, pigs and the like, the pathogenesis and treatment methods of myocardial infarction were developed. In particular, since pigs have very similar characteristics to humans in physiology, anatomical structure and cardiovascular structure, they are animals widely used in non-clinical experiments on large animals, which are essential steps before conducting clinical trials. In general, a porcine myocardial infarction model is produced by inducing coronary arterial reperfusion, or using surgical methods. However, these methods have disadvantages of having a high mortality rate and taking a lot of time when inducing myocardial infarction, and causing severe infection and pericarditis after surgery. In addition, in case of porcine animal models, there is a limitation in that young individuals with rapid recovery should be used due to technological and environmental factors, which leads to limitation in securing valid data. Therefore, there is a need to develop a new preparation method of an animal model which has a low mortality rate after inducing myocardial infarction, and can induce myocardial infarction for a long period of time, and the method of preparation of a myocardial infarction model using 3D bioprinting can be determined as an optimal technology to meet this need.

### DISCLOSURE

### Technical Problem

One embodiment of the present invention relates to a biocompatible polymer-based customized cylindrical hollow construct (implant) capable of inducing partial blood flow compression in coronary arteries using 3-dimensional printing technology and a method for preparation thereof.

Another embodiment of the present invention relates to a method for inducing myocardial infarction, which induces myocardial infarction by implanting the hollow construct into a target blood vessel to partially block blood flow.

Other embodiment of the present invention relates to a large animal-based myocardial infarction model in which the hollow construct is applied to a minimal surgical method and a method for preparation thereof.

### Technical Solution

The present invention has established a process condition of a customized implant for preparing animal experiment models each having different heart structures, and established verification of effectiveness of a large animal myocardial infarction model through angiocardiography, echocardiography, histologic lesion examination, and the like.

Hereinafter, the present invention will be described in more detail.

One embodiment of the present invention relates to a cylindrical hollow construct with a through-hole extended in a longitudinal direction, more specifically, a hollow construct for being implanted to a blood vessel for inducing myocardial infarction, which is implanted into a target blood vessel and induces myocardial infarction, and it may have an inner diameter with a certain size so that blood flow can pass therethrough. The hollow construct may be a block apparatus for blood vessel or occluder apparatus for blood vessel capable of inducing myocardial infarction by partially blocking or occluding blood flow or blood vessel, as being implanted into the blood vessel.

The hollow construct may be implanted into a blood vessel on purpose of inducing myocardial infarction, and the hollow construct may be implanted to a blood vessel that can induce myocardial infarction if blood flow is blocked. For example, the hollow construct according to one embodiment of the present invention may be implanted into one or more selected from the group consisting of a left anterior descending artery, a right coronary artery and a left circumflex and induce myocardial infarction.

The implantable hollow construct for inducing myocardial infarction according to one embodiment of the present invention is implanted into a target blood vessel and induce myocardial infarction by partial blockage of blood flow. At this time, when blood flow is completely blocked or blocked over a threshold, an individual in which the hollow construct is implanted may die by blockage of blood flow, and therefore, it may be formed to have an inner diameter of a certain size so as to prevent death of the individual and induce myocardial infarction. In other words, the inner diameter of the hollow construct may be formed to be smaller than a certain size to partially block blood flow, and for example, the inner diameter of the hollow construct may be formed to be 90% or less, 80% or less, 70% or less, 60% or less, 50% or less, 40% or less, 30% or less, 28% or less, 25% or less, 20% or less, or 15% or less of the outer diameter of the hollow construct. In addition, the inner diameter of the hollow construct may be formed over a certain size so that the individual does not die by excessively blocking blood flow, and even if the lower limit of the inner diameter of the hollow construct is not specified, those skilled in the art will be able to set an appropriate inner diameter of the hollow construct so that the individual does not die and myocardial infarction is induced, and for example, the inner diameter of the hollow construct may be formed to be 1% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, or 80% or more of the outer diameter of the hollow construct, but not limited thereto. Then, the inner diameter of the hollow construct may be set in combination of the upper limit and the lower limit based on the outer diameter of the hollow construct.

The hollow construct may have an outer diameter with a certain size so as to be implanted into a target blood vessel, and for example, the outer diameter of the hollow construct may range 70% to 130%, 70% to 125%, 70% to 120%, 70% to 115%, 70% to 110%, 70% to 105%, 75% to 130%, 75% to 125%, 75% to 120%, 75% to 115%, 75% to 110%, 75% to 105%, 80% to 130%, 80% to 125%, 80% to 120%, 80% to 115%, 80% to 110%, 80% to 105%, 85% to 130%, 85% to 125%, 85% to 120%, 85% to 115%, 85% to 110%, 85% to 105%, 90% to 130%, 90% to 125%, 90% to 120%, 90% to 115%, 90% to 110%, 90% to 105%, 95% to 130%, 95% to 125%, 95% to 120%, 95% to 115%, 95% to 110%, 95% to 105%, 100% to 130%, 100% to 125%, 100% to 120%, 100% to 115%, 100% to 110%, 100% to 105%, 105% to 130%, 105% to 125%, 105% to 120%, 105% to 115%, 105% to 110%, 110% to 130%, 110% to 125%, 110% to 120%, 110% to 115%, 115% to 130%, 115% to 125%, 115% to 120%, 120% to 130%, 120% to 125%, or 125% to 130% of the inner diameter of the target blood vessel, and as one example, the outer diameter of the hollow construct may range 70% to 130% of the inner diameter of the target blood vessel.

As one example, the outer diameter of the hollow construct may be 1 to 4 mm, 1 to 3.5 mm, 1 to 3 mm, 1 to 2.5 mm, 1 to 2 mm, 1.5 to 4 mm, 1.5 to 3.5 mm, 1.5 to 3 mm, 1.5 to 2.5 mm, or 1.5 to 2 mm, but not limited thereto, and as afore-mentioned, the outer diameter of the hollow construct according to one embodiment of the present invention may be formed to have a certain size so as to be implanted into a target blood vessel to partially block blood flow.

As one example, the inner diameter of the hollow construct may be 0.1 to 2 mm, 0.1 to 1.5 mm, 0.1 to 1 mm, 0.1 to 0.5 mm, 0.2 to 2 mm, 0.2 to 1.5 mm, 0.2 to 1 mm, or 0.2 to 0.5 mm but not limited thereto, and as afore-mentioned, the inner diameter of the hollow construct according to one embodiment of the present invention may be formed to have an inner diameter with certain size so as to block blood flow enough to induce myocardial infarction and prevent death of an individual.

As one example, the hollow construct may be formed in length of 1.5 to 5 mm, 1.5 to 4.5 mm, 1.5 to 4 mm, 1.5 to 3.5 mm, 1.5 to 3 mm, 2 to 5 mm, 2 to 4.5 mm, 2 to 4 mm, 2 to 3.5 mm, 2 to 3 mm, 2.5 to 5 mm, 2.5 to 4.5 mm, 2.5 to 4 mm, 2.5 to 3.5 mm, or 2.5 to 3 mm, but not limited thereto.

The implantable hollow construct for inducing myocardial infarction according to one embodiment of the present invention may be a biodegradable polymer material. For example, the material of the hollow construct may be one or more selected from the group consisting of polycaprolactone (PCL), poly(lactate-co-glycolate) (PLGA), poly lactic acid (PLA), polyurethane (PU), poly(lactide-co-caprolactone) (PLCL), polydioxanone (PDO), polystyrene (PS), poly(ethylene glycol) (PEG), poly(vinyl acetate) (PVA), polypropylene glycol (PPG), polyacrylamide (PAAm), polyglycolic acid (PGA), polymethylmethacrylate (PMMA), polyhydroxybutyrate (PHB), and polyvinylpyrrolidone (PVP), but not limited thereto.

Another embodiment of the present invention relates to a method for production of a implantable hollow construct for inducing myocardial infarction, comprising measuring an inner diameter of a target blood vessel; preparing a polymer melt; and 3-dimensional printing of the polymer melt into a cylindrical hollow construct form having an inner diameter with a certain size in which a through-hole extended in a longitudinal direction is formed. The target blood vessel may be a blood vessel of an animal other than humans.

The target blood vessel may be selected as a blood vessel in which myocardial infarction may be induced when blood flow is blocked, and for example, it may be one or more selected from the group consisting of a left anterior descending artery, a right coronary artery and a left circumflex artery to induce myocardial infarction.

The 3-dimensional printing of the polymer melt may be printing so that the outer diameter of the hollow construct is within a range of 70% to 130%, 70% to 125%, 70% to 120%, 70% to 115%, 70% to 110%, 70% to 105%, 75% to 130%, 75% to 125%, 75% to 120%, 75% to 115%, 75% to 110%, 75% to 105%, 80% to 130%, 80% to 125%, 80% to 120%, 80% to 115%, 80% to 110%, 80% to 105%, 85% to 130%, 85% to 125%, 85% to 120%, 85% to 115%, 85% to 110%, 85% to 105%, 90% to 130%, 90% to 125%, 90% to 120%, 90% to 115%, 90% to 110%, 90% to 105%, 95% to 130%, 95% to 125%, 95% to 120%, 95% to 115%, 95% to 110%, 95% to 105%, 100% to 130%, 100% to 125%, 100% to 120%, 100% to 115%, 100% to 110%, 100% to 105%, 105% to 130%, 105% to 125%, 105% to 120%, 105% to 115%, 105% to 110%, 110% to 130%, 110% to 125%, 110% to 120%, 110% to 115%, 115% to 130%, 115% to 125%, 115% to 120%, 120% to 130%, 120% to 125%, or 125% to 130% of the inner diameter of the target blood vessel, and as one example, it may be printing so that the outer diameter of the hollow construct is within a range of 70% to 130% of the inner diameter of the target blood vessel.

The 3-dimensional printing of the polymer melt may be printing so that the inner diameter of the hollow construct is to be 90% or less, 80% or less, 70% or less, 60% or less, 50% or less, 40% or less, 30% or less, 28% or less, 25% or less, 20% or less, or 15% or less of the outer diameter of the hollow construct.

The 3-dimensional printing of the polymer melt may be printing so that the outer diameter of the hollow construct is to be 1 to 4 mm, 1 to 3.5 mm, 1 to 3 mm, 1 to 2.5 mm, 1 to 2 mm, 1.5 to 4 mm, 1.5 to 3.5 mm, 1.5 to 3 mm, 1.5 to 2.5 mm, or 1.5 to 2 mm, but not limited thereto, and as aforementioned, it may be printing so that the hollow construct according to one embodiment of the present invention has a certain size so as to be implanted into a target blood vessel to partially block blood flow.

The 3-dimensional printing of the polymer melt may be printing so that the inner diameter of the hollow construct is to be 0.1 to 2 mm, 0.1 to 1.5 mm, 0.1 to 1 mm, 0.1 to 0.5 mm, 0.2 to 2 mm, 0.2 to 1.5 mm, 0.2 to 1 mm, or 0.2 to 0.5 mm, but not limited thereto, and as aforementioned, it may be printing so that the hollow construct according to one embodiment of the present invention has an inner diameter with a certain size to block blood flow so as to induce myocardial infarction and prevent death of an individual.

The 3-dimensional printing of the polymer melt may be printing so that the length of the hollow construct has a length of 1.5 to 5 mm, 1.5 to 4.5 mm, 1.5 to 4 mm, 1.5 to 3.5 mm, 1.5 to 3 mm, 2 to 5 mm, 2 to 4.5 mm, 2 to 4 mm, 2 to 3.5 mm, 2 to 3 mm, 2.5 to 5 mm, 2.5 to 4.5 mm, 2.5 to 4 mm, 2.5 to 3.5 mm, or 2.5 to 3 mm, but not limited thereto.

Other one embodiment of the present invention relates to a method for inducing myocardial infarction, comprising measuring an inner diameter of a target blood vessel; preparing a cylindrical hollow construct with a through-hole extended in a longitudinal direction, which has an inner diameter with certain size allowing blood flow to pass through, by 3-dimensional printing a polymer melt; and inducing myocardial infarction by implanting the hollow construct into the target blood vessel to partially block blood flow. The target blood vessel may be a blood vessel of an animal other than humans. The outer diameter of the hollow construct may have an outer diameter with certain size so as to be implanted into a target blood vessel, and the outer diameter of the hollow construct based on the inner diameter of the target blood vessel is as aforementioned, and for example, the hollow construct may have an outer diameter within a range of 70% to 130% of the inner diameter of the target blood vessel.

The animal may be a mammal other than humans, and for example, the animal may be one or more selected from the group consisting pigs, monkeys, chimpanzees, sheep, goats, cows, horses, camels, dogs and cats, but not limited thereto.

The target blood vessel may be selected as a blood vessel in which myocardial infarction may be induced when blood flow is blocked, and for example, it may be at least one selected from the group consisting of a left anterior descending artery, a right coronary artery and a left circumflex artery to induce myocardial infarction.

The preparing the cylindrical hollow construct may prepare it so that the inner diameter of the cylindrical hollow construct is to be 90% or less, 80% or less, 70% or less, 60% or less, 50% or less, 40% or less, 30% or less, 28% or less, 25% or less, 20% or less, or 15% or less of the outer diameter of the cylindrical hollow construct. In addition, the preparing the cylindrical hollow construct may prepare it so that the cylindrical hollow construct has an inner diameter of a certain size or more so that an individual does not die by excessively blocking blood flow, and for example, may prepare it so that the inner diameter of the hollow construct is to be 1% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, or 80% or more of the outer diameter of the hollow construct, but not limited thereto. Then, the preparing the cylindrical hollow construct may prepare it so that the inner diameter of the hollow construct is set in combination of the upper limit and lower limit based on the outer diameter of the hollow construct.

The preparing the cylindrical hollow construct may prepare it so that the outer diameter of the hollow construct is in a size of 1 to 4 mm, 1 to 3.5 mm, 1 to 3 mm, 1 to 2.5 mm, 1 to 2 mm, 1.5 to 4 mm, 1.5 to 3.5 mm, 1.5 to 3 mm, 1.5 to 2.5 mm, or 1.5 to 2 mm, but not limited thereto, and as aforementioned, it may prepare it so that the hollow construct according to one embodiment of the present invention has a certain size to be implanted into a target blood vessel to partially block blood flow.

The preparing the cylindrical hollow construct may prepare it so that the inner diameter of the hollow construct is 0.1 to 2 mm, 0.1 to 1.5 mm, 0.1 to 1 mm, 0.1 to 0.5 mm, 0.2 to 2 mm, 0.2 to 1.5 mm, 0.2 to 1 mm, or 0.2 to 0.5 mm, but not limited thereto, and as aforementioned, it may prepare it so that the hollow construct according to one embodiment of the present invention has an inner diameter with certain size to block blood flow so as to prevent death of an individual and induce myocardial infarction.

The preparing the cylindrical hollow construct may prepare the length of the cylindrical hollow construct is in a length of 1.5 to 5 mm, 1.5 to 4.5 mm, 1.5 to 4 mm, 1.5 to 3.5 mm, 1.5 to 3 mm, 2 to 5 mm, 2 to 4.5 mm, 2 to 4 mm, 2 to 3.5 mm, 2 to 3 mm, 2.5 to 5 mm, 2.5 to 4.5 mm, 2.5 to 4 mm, 2.5 to 3.5 mm, or 2.5 to 3 mm, but not limited thereto.

The implanting may be implanting the cylindrical hollow construct into a left anterior descending artery by injecting it into a carotid artery.

The method for inducing myocardial infarction according to one embodiment of the present invention may not comprise removing the cylindrical hollow construct implanted into the target blood vessel. Specifically, while the conventional method for inducing myocardial infarction using a balloon catheter may cause unintended physical damages of blood vessels or tissues and show a low survival rate in the process of temporarily blocking blood flow inside an artery and then removing the balloon catheter again, the method for inducing myocardial infarction according to one embodiment of the present invention can induce myocardial infarction with high stability and achieve a high survival rate as it does not comprise removing the cylindrical hollow construct after implantation (Example 4).

The method for inducing myocardial infarction according to one embodiment of the present invention may further comprise breeding an animal in which the hollow construct is implanted.

The method for inducing myocardial infarction according to one embodiment of the present invention may induce one or more of the following (1) to (6) as the hollow construct is implanted into an animal:

(1) reduction of a left anterior descending artery diameter

(2) reduction of ejection fraction

(3) necrosis of cardiac tissue

(4) abnormal morphological changes in cardiomyocytes

(5) collagen accumulation inside myocardial tissue

(6) induction of hypoxia of myocardial tissue.

In the method for inducing myocardial infarction according to one embodiment of the present invention, the survival rate (%) of the animal in 5 weeks after implanting the hollow construct is over 50%, 55% or more, 60% or more, 70% or more, 75% or more, 80% or more, 85% or more, or 90% or more. Then, even though the upper limit of the survival rate (%) is not specified, those skilled in the art will be able to clearly understand the technical effect of the present invention, in which myocardial infarction is induced to the extent, but for example, the upper limit of the survival rate (%) may be 100% or less, 99% or less, 95% or less, or 90% or less, but not limited thereto.

In the method for inducing myocardial infarction according to one embodiment of the present invention, the necrosis rate (%) of the heart tissue (for example, left ventricle tissue) in 6 weeks after implanting the hollow construct may be 15% or more, 20% or more, 24% or more, 25% or more, or 30% or more. Then, even though the upper limit of the necrosis rate (%) of the heart tissue is not specified, those skilled in the art will be able to clearly understand the effect of the present invention in which myocardial infarction is induced by the cylindrical hollow construct, but for example, the upper limit of the necrosis rate (%) of the heart issue may be 100% or less, 95% or less, 90% or less, 85% or less, 80% or less, 75% or less, 70% or less, 65% or less, 60% or less, 55% or less, or 50% or less, but not limited thereto.

In the method for inducing myocardial infarction according to one embodiment of the present invention, the reduction rate (%) of the ejection fraction (for example, ejection fraction of left ventricle) in 6 weeks after implanting the hollow construct may be 20% or more, 25% or more, 30% or more, 35% or more, or 40% or more. The reduction rate (%) of the ejection fraction in 6 weeks after implantation is a value derived by dividing the difference between the average ejection fraction before implanting the hollow construct and the average ejection fraction in 6 weeks after implanting the hollow construct by the average ejection fraction before implanting the hollow construct. Then, even though the upper limit of the reduction rate of the ejection fraction is not specified, those skilled in the art will be able to clearly understand the effect of the present invention in which myocardial infarction is induced by the cylindrical hollow construct, but for example, the upper limit of the reduction rate (%) of the ejection fraction may be 100% or less, 95% or less, 90% or less, 85% or less, 80% or less, 75% or less, 70% or less, 65% or less, 60% or less, 55% or less, or 50% or less, but not limited thereto.

In the method for inducing myocardial infarction according to one embodiment of the present invention, the reduction rate (%) of the left anterior descending artery inner diameter in 6 weeks after implanting the hollow construct may be 20% or more, 30% or more, 40% or more, or 45% or more. The reduction rate (%) of the left anterior descending artery in 6 weeks after implanting is a value derived by dividing the difference between the average left anterior descending artery inner diameter before implanting the hollow construct and the average left anterior descending artery inner diameter in 6 weeks after implanting the hollow construct by the average left anterior descending artery inner diameter before implanting the hollow construct. Then, even though the upper limit of the reduction rate of the left anterior descending artery inner diameter is not specified, those skilled in the art will be able to clearly understand the effect of the present invention in which myocardial infarction is induced by the cylindrical hollow construct, but for example, the upper limit of the reduction rate (%) of the left anterior descending artery inner diameter may be 100% or less, 95% or less, 90% or less, 85% or less, 80% or less, 75% or less, 70% or less, 65% or less, 60% or less, 55% or less, or 50% or less, but not limited thereto.

Other one embodiment of the present invention relates to an animal model with myocardial infraction other than humans, comprising an implantable hollow construct for inducing myocardial infraction into a target blood vessel, in which the implantable hollow construct for inducing myocardial infarction is a cylindrical hollow construct having an inner diameter with certain size and a through-hole extended in a longitudinal direction is formed. The outer diameter of the hollow construct may be an outer diameter with a certain size so as to be implanted into a target blood vessel, and the outer diameter of the hollow construct based on the inner diameter of the target blood vessel is as aforementioned, and for example, the hollow construct may have an outer diameter in a range of 70% to 130% of the inner diameter of the target blood vessel.

The animal may be a mammal other than humans, and for example, the animal may be at least one selected from the group consisting pigs, monkeys, chimpanzees, sheep, goats, cows, horses, camels, dogs and cats, but not limited thereto.

The target blood vessel may be selected as a blood vessel in which myocardial infarction may be induced when blood flow is blocked, and for example, it may be at least one selected from the group consisting of a left anterior descending artery, a right coronary artery and a left circumflex artery to induce myocardial infarction.

The animal model according to one embodiment of the present invention may induce one or more of the following (1) to (6):

(1) reduction of a left anterior descending artery diameter

(2) reduction of ejection fraction

(3) necrosis of cardiac tissue

(4) abnormal morphological changes in cardiomyocytes

(5) collagen accumulation inside myocardial tissue

(6) induction of hypoxia of myocardial tissue.

In the animal model according to one embodiment of the present invention, the survival rate in 5 weeks after the hollow construct is implanted is over 50%, 55% or more, 60% or more, 70% or more, 75% or more, 80% or more, 85% or more, or 90% or more. Then, even though the upper limit of the survival rate is not specified, those skilled in the art will be able to clearly understand the effect of the present invention in which myocardial infarction is induced to the extent that an animal model in which the cylindrical hollow construct is implanted does not die, but for example, the upper limit of the survival rate (%) may be 100% or less, 99% or less, 95% or less, or 90% or less, but not limited thereto.

In the animal model according to one embodiment of the present invention, the necrosis rate (%) of the heart tissue (for example, left ventricle tissue) in 6 weeks after implanting the hollow construct may be 15% or more, 20% or more, 24% or more, 25% or more, or 30% or more. Then, the upper limit of the necrosis rate (%) of the heart tissue is as aforementioned.

In the animal model according to one embodiment of the present invention, the reduction rate (%) of the ejection rate (for example, ejection fraction of left ventricle) in 6 weeks after implanting the hollow construct may be 20% or more, 25% or more, 30% or more, 35% or more, or 40% or more. The reduction rate (%) of the ejection fraction in 6 weeks after implantation and the upper limit of the reduction rate of the ejection fraction are as aforementioned.

In the animal model according to one embodiment of the present invention, the reduction rate (%) of the left anterior descending artery inner diameter in 6 weeks after implanting the hollow construct may be 20% or more, 30% or more, 40% or more, or 45% or more. The reduction rate of the left anterior descending artery inner diameter in 6 weeks after implantation and the upper limit of the reduction rate of the left anterior descending artery inner diameter are as aforementioned.

Other one embodiment of the present invention relates to a method for preparation of an animal model with myocardial infarction other than humans, comprising inducing myocardial infarction of the animal by implanting an implantable hollow construct for inducing myocardial infarction into the inside of a target blood vessel of an animal, wherein the implantable hollow construct for inducing myocardial infarction is a cylindrical hollow construct having an inner diameter with a certain size and a through-hole extended in a longitudinal direction is formed to allow blood flow to pass through. The outer diameter of the hollow construct may be an outer diameter with a certain size so as to be implanted into a target blood vessel, and the outer diameter of the hollow construct based on the inner diameter of the target blood vessel is as aforementioned, and for example, the hollow construct may have an outer diameter in a range of 70% to 130% of the inner diameter of the target blood vessel.

The implanting may be implanting the hollow construct into a left anterior descending artery by injecting it into a carotid artery.

The method for preparation of an animal model with myocardial infarction according to one embodiment of the present invention may not comprise removing the cylindrical hollow construct implanted in the target blood vessel.

In the method for preparation of an animal model with myocardial infarction according to one embodiment of the present invention, the survival rate of the animal in 5 weeks after implanting the hollow construct may be over 50%, 55% or more, 60% or more, 70% or more, 75% or more, 80% or more, 85% or more, or 90% or more.

The method for preparation of an animal model with myocardial infarction according to one embodiment of the present invention may further comprise breeding an animal in which the hollow construct is implanted.

The hollow construct may induce one or more of the following (1) to (6) as implanted into an animal:

(1) reduction of a left anterior descending artery diameter

(2) reduction of ejection fraction

(3) necrosis of cardiac tissue

(4) abnormal morphological changes in cardiomyocytes

(5) collagen accumulation inside myocardial tissue

(6) induction of hypoxia of myocardial tissue.

Other one embodiment of the present invention relates to a screening method of a myocardial infarction prevention or treatment substance, comprising administering a candidate substance for prevention or treatment of myocardial infarction to an animal comprising the hollow construct according to the present invention; and selecting the candidate substance as a prevention or treatment substance of myocardial infarction, when myocardial infarction of the animal model is improved after administering the candidate substance.

Other one embodiment of the present invention relates to a screening method of a myocardial infarction prevention or treatment substance, comprising administering a candidate substance for prevention or treatment of myocardial infarction to an animal in which myocardial infarction is induced by the method for inducing myocardial infarction according to the present invention; and selecting the candidate substance as a prevention or treatment substance of myocardial infarction, when myocardial infarction of the animal model is improved after administering the candidate substance.

Other one embodiment of the present invention relates to a screening method of a myocardial infarction prevention or treatment substance, comprising administering a candidate substance for prevention or treatment of myocardial infarction to the animal model with myocardial infarction according to the present invention; and selecting the candidate substance as a prevention or treatment substance of myocardial infarction, when myocardial infarction of the animal model is improved after administering the candidate substance.

Other one embodiment of the present invention relates to a screening method of a myocardial infarction prevention or treatment substance, comprising administering a candidate substance for prevention or treatment of myocardial infarction to an animal model prepared by the method for preparation of an animal model with myocardial infarction according to the present invention; and selecting the candidate substance as a prevention or treatment substance of myocardial infarction, when myocardial infarction of the animal model is improved after administering the candidate substance.

The improvement of myocardial infarction may be due to one or more of the following (1) to (7):

(1) maintenance or increase of a left anterior descending artery diameter

(2) maintenance or increase of ejection fraction

(3) alleviation or delay of cardiac tissue necrosis

(4) alleviation or delay of abnormal morphological changes of cardiomyocytes

(5) alleviation or delay of collagen accumulation inside myocardial tissue

(6) alleviation or improvement of hypoxia of myocardial tissue

(7) increase in survival rate compared to a control group not administered with the candidate substance.

### Advantageous Effects

When myocardial infarction occurs, plaque is formed inside an artery, which makes normal blood flow to the myocardium impossible, and the myocardium does not receive oxygen and nutrients, and therefore, necrosis is induced. Conventional methods induce myocardial infarction by a method of including myocardial damage by temporarily blocking blood flow inside a normal artery, and then releasing blood flow blockage again, in order to imitate this. For example, a representative method for preparing a conventional myocardial infarction model is a method of temporarily blocking blood flow inside an artery using a balloon catheter.

However, such a conventional method is difficult to say that it imitates the actually formed myocardial infarction environment, and also, it had a problem in that a lot of costs and time were consumed to produce a myocardial infarction model, as it causes low survival rate, low myocardial necrosis rate, unstable ejection fraction and the like. In order to solve this conventional problem, the method suggested in the present invention can reproduce an environment similar to actual myocardial infarction by realizing permanent partial blocking of blood flow, and by showing improved cardiac function deterioration according thereto, production of an effective myocardial infarction inducing model was possible.

The present invention can successively imitate myocardial infarction by partially blocking blood flow permanently using a biocompatible polymer, and can produce a cylindrical form of complex construct having a through-hole using 3D bioprinting technology, and therefore, it has an excellent effect compared to the conventional animal model production method in terms of time, reproducibility, customization, production efficiency, and the like.

### BRIEF DESCRIPTION OF THE DRAWINGS

FIG. 1a is a drawing which shows the overall view of the 3-dimensional bioprinting system according to one embodiment of the present invention.

FIG. 1B is a drawing which shows the shapes and outer diameter and inner diameter of the hollow constructs for inducing myocardial infarction in various sizes prepared according to one embodiment of the present invention.

FIG. 1c is a schematic diagram which shows the process of injection of the hollow construct for inducing myocardial infarction placed in a coronary artery according to one embodiment of the present invention.

FIG. 1d is a drawing which shows the figure of the front portion and side portion of the hollow construct for inducing myocardial infarction according to one embodiment of the present invention.

a of FIG. 2 is a drawing which shows the initial cardiac angiography result of the animal model in which the hollow construct for inducing myocardial infarction according to one embodiment of the present invention is implanted, and b of FIG. 2 is a drawing which shows the cardiac angiography result after placing the hollow construct, and the position of the hollow construct is indicated by a white arrow, and c of FIG. 2 is a drawing which shows the cardiac angiography result which confirms partial blood flow blocking induced by the hollow construct.

FIG. 3a is a graph which shows the change in the coronary artery inner diameter by the hollow construct for inducing myocardial infarction according to one embodiment of the present invention.

FIG. 3b is a graph which shows the change in the ejection fraction of left ventricle by the hollow construct for inducing myocardial infarction according to one embodiment of the present invention.

FIG. 4 is a drawing which shows the result of observing the change in the left ventricle wall after in 6 weeks after inducing myocardial infarction according to one embodiment of the present invention through various biopsy methods (Bar: 100 μm), and a of FIG. 4 is a drawing which shows the TTC staining result, and b and e of FIG. 4 are drawings which show the H&E staining result, and c and f of FIG. 4 are drawings which show the picrosirius red staining result, and d and g of FIG. 4 are drawings which show the immunohistochemical staining result.

### MODE FOR INVENTION

Hereinafter, the present invention will be described in more detail by the following examples. However, these examples are intended to illustrate the present invention only, but the scope of the present invention is not limited by these examples.

### Example 1: Production of Hollow Constructs for Inducing Myocardial Infarction

(1) Production of Hollow Constructs Having Various Outer Diameters and Inner Diameters

The hollow construct for inducing myocardial infarction according to one embodiment of the present invention can be produced by various materials according to the purpose, and in the present example, illustratively, a PCL (Polycaprolactone) polymer with excellent biocompatibility was used as a material.

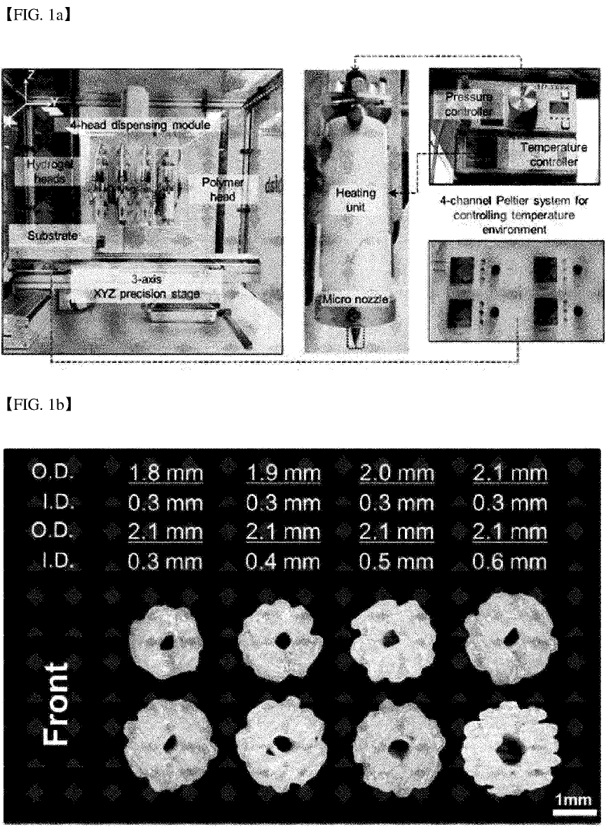

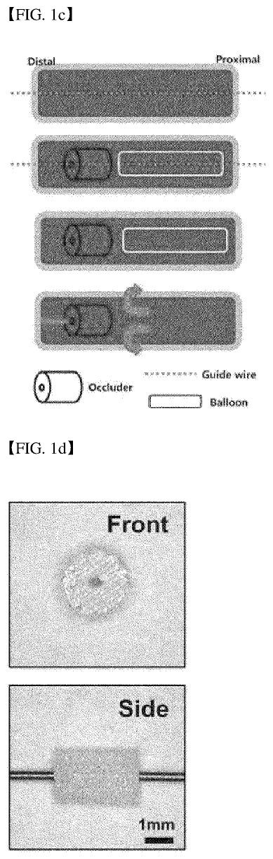

The PCL polymer melt was injected at the polymer extrusion head at the top of the 3D bioprinting system capable of temperature and pressure control under the condition of the syringe internal temperature of 70° C. and pneumatic pressure of 550 kPa, and the PCL melt was extruded into a micro-size through a nozzle with a dimeter of 150 μm to prepare cylindrical hollow constructs having a through-hole in the center were prepared in various sizes. A nozzle transfer path for the cylindrical hollow constructs was produced using a G-code production program, and a continuous and stable hollow construct production process was established by giving a stage transfer rate of 60 mm/min during polymer extrusion (FIG. 1a). FIG. 1a is a drawing which shows the overall view of the 3-dimensional bioprinting system according to one embodiment of the present invention.

Myocardial infarction is disease that generally occurs when blood flow is blocked due to deformation inside the left anterior descending artery (LAD) and sufficient oxygen and nutrients are not received therefrom, and therefore, in order to imitate the environment where myocardial infarction occurs, a hole was formed in the center of the implant, and the internal shape of the coronary artery and the hollow construct form which minimizes damages from blood pressure were considered, and it was possible to customize with various sizes of outer diameters and inner diameters, so it was possible to block unexpected variables that might occur in an animal experiment (FIG. 1B). FIG. 1B is a drawing which shows the form and outer diameter and inner diameter of the hollow constructs with various sizes prepared according to one embodiment of the present invention. As shown in FIG. 1B, the length of the hollow construct was prepared as 3 mm, and the outer diameter and inner diameter of the hollow construct could be controlled up to a 0.1 mm unit. The conventional myocardial infarction model production method induced myocardial infarction by a method of temporarily blocking blood flow by inserting a catheter into an artery to induce myocardial damage and then removing the catheter again, but in the process of removing the catheter, unintentional physical damage to blood vessels or tissues may occur, which may lead to a decrease in the survival rate, and due to the nature of the implant to be inserted into a very thin blood vessel, even a light change in the size of the implant may affect induction of myocardial infarction. Therefore, the method for production according to one embodiment of the present invention could finely adjust the size of the implant for inducing myocardial infarction and be individual-specifically customized, and thus, could induce myocardial infarction with high stability.

(2) Production of Hollow Construct Customized to Target Blood Vessel

Seven three-month-old Yorkshire×Landrace F1 crossbred castrated male pigs were prepared, and the inner diameters of left anterior descending arteries were measured through quantitative coronary angiography. The measured inner diameters of the left anterior descending artery of the 7 pigs were shown in Table 1.

TABLE 1

| | Left anterior | | |
|---|---|---|---|
| | descending | | |
| | artery inner | Implant outer | Implant inner |
| Sample NO. | diameter (mm) | diameter (mm) | diameter (mm) |
| 1 | 1.94 | 2.10 | 0.30 |
| 2 | 2.1 | 2.40 | 0.30 |
| 3 | 2.13 | 2.10 | 0.30 |
| 4 | 1.71 | 1.80 | 0.50 |
| 5 | 2.05 | 1.80 | 0.50 |
| 6 | 2.35 | 2.40 | 0.30 |
| 7 | 1.98 | 2.10 | 0.30 |
| Standard | 0.20 | — | — |
| deviation | | | |

As shown in Table 1, considering that the standard deviation of the inner diameter of the left anterior descending artery of each individual was shown as about 0.20 mm, and the average inner diameter of the left anterior descending artery was very thin as about 2 mm, the deviation was very large, so it could be found that it was necessary to specifically prepare an implant for inducing myocardial infarction for each individual. Accordingly, the outer diameter and inner diameter of the implant was prepared suitable for the left anterior descending artery inner diameter of each individual shown in Table 1, and the outer diameter and inner diameter of the hollow construct specific to each individual were shown in Table 1. The outer diameter of the hollow construct was set to be 70% to 130% of the value based on the inner diameter of the left anterior descending artery of each individual, and the inner diameter was set to be 10% to 40% of the implant outer diameter considering the probability of death of each individual and appropriateness of myocardial induction that could be induced by partial blood flow blockage.

### Example 2: Implantation of Hollow Construct for Inducing Myocardial Infarction and Production of Animal Model

The hollow construct for inducing myocardial infarction specific to an individual prepared in Example 1 was fixed to the center of the guide wire at the top of the balloon expansion catheter and implanted so as to be placed from the carotid artery to the left anterior descending artery (LAD) of each individual (FIG. 1c and FIG. 1d). This implantation method could achieve a high survival rate after inducing myocardial infarction, by minimizing a surgical process and partially blocking generated blood flow. FIG. 1c is a schematic diagram which shows the process of injection of the hollow construct placed to a coronary artery, and FIG. 1d is a drawing which shows the figure of the front portion and side portion of the hollow construct.

### Example 3: Confirmation of Myocardial Infarction Induction of Animal Model with Myocardial Infarction

(1) Observation of Blood Flow Inside Coronary Artery

In order to confirm whether the hollow construct prepared in Example 1 performs substantial blood flow blockage in the animal model with myocardial infarction, an angiocardiography test was performed before implantation of the hollow construct (a of FIG. 2), immediately after follow implantation of the hollow construct (b of FIG. 2) and in 5 weeks after implantation of the hollow construct (c of FIG. 2), respectively, and the blood flow inside the coronary artery was observed and shown in FIG. 2. As indicated by a white arrow in b of FIG. 2, the hollow construct used in the experiment was placed accurately on the position of the left anterior descending arteries of all pigs. As shown in c of FIG. 2, by inducing partial blood flow up to 5 weeks after implantation, stable myocardial infarction was successfully induced.

(2) Observation of Change in Heart Function

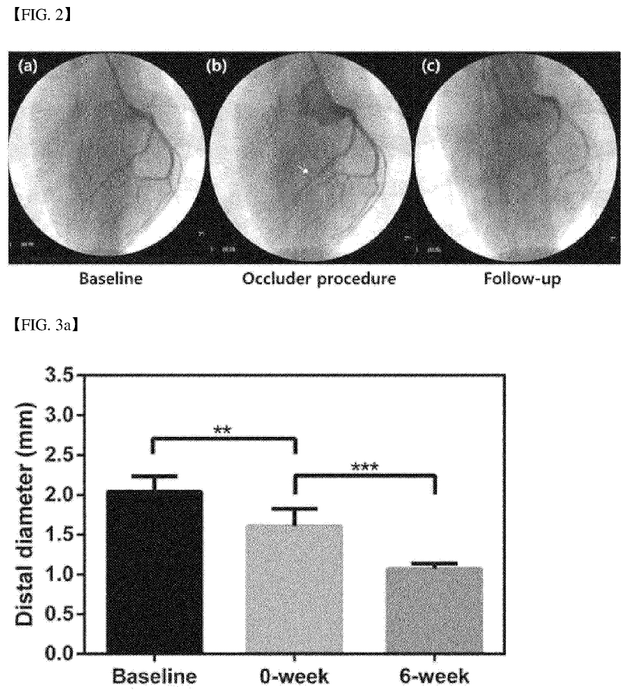

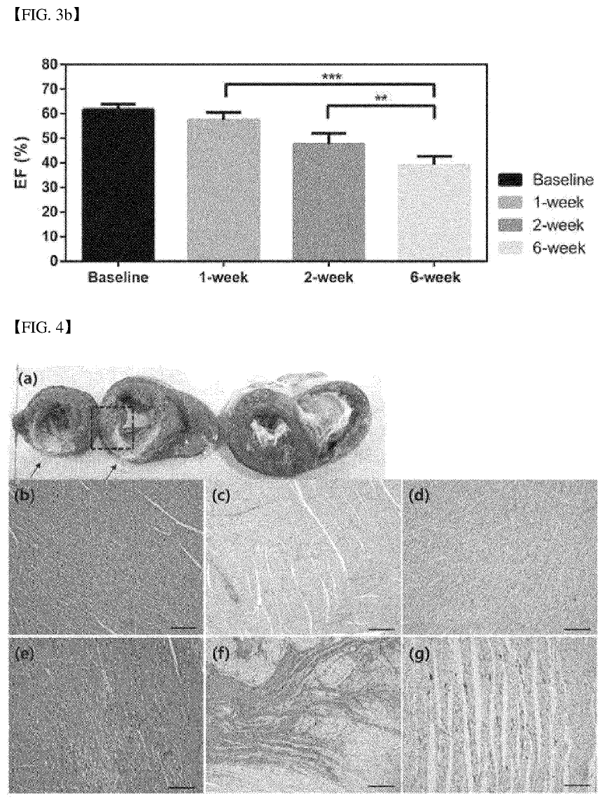

The change in heart function due to the hollow construct was observed through echocardiography and quantitative coronary angiography. Table 2 shows the change in the coronary artery inner diameter after implanting the hollow construct according to one embodiment of the present invention, and this was graphed and shown in FIG. 3a. As shown in Table 2 and FIG. 3a, the inner diameter of the left anterior descending artery was significantly reduced over time after applying the hollow construct. In addition, Table 3 shows the change in the ejection fraction of the left ventricle after implanting the hollow construct according to one embodiment of the present invention, and this was graphed and shown in FIG. 3b. As shown in Table 3 and FIG. 3b, the most important index for evaluating heart function, the ejection fraction (EF) was also reduced by about 30% in 6 weeks after implanting the hollow construct, showing the numerical value of severe myocardial infarction was shown, and quantitative suitability of the large animal-based myocardial infarction model produced by the 3-dimensional bioprinting technology according to one embodiment of the present invention was verified. In FIG. 3a and FIG. 3b, the baseline means the blood vessel inner diameter before implantation.

TABLE 2

| | Left anterior descending | Left anterior descending |
|---|---|---|
| | artery inner | artery inner diameter |
| Classification | diameter (mm) | reduction rate (%) |
| Baseline (before | 2.04 ± 0.18 | — |
| implantation) | | |
| Immediately after | 1.61 ± 0.20 | 21.08 |
| implantation | | |
| In 6 weeks after | 1.08 ± 0.05 | 47.06 |
| implantation | | |

TABLE 3

| | Ejection | Ejection fraction |
|---|---|---|
| Classification | fraction (%) | reduction rate (%) |
| Baseline (before implantation) | 67.98 ± 3.81 | — |
| In 1 week after implantation | 52.40 ± 4.35 | 22.92 |
| In 2 weeks after implantation | 45.41 ± 4.45 | 33.20 |
| In 6 weeks after implantation | 39.09 ± 3.31 | 42.50 |

(3) Histopathological Test

Through a histopathological test, whether the method for inducing myocardial infarction according to one embodiment of the present invention successfully induced myocardial infarction was confirmed. The histopathological test is classified into 4 kinds of staining methods such as histological test of TTC (Triphenyltetrazolium chloride), H&E (Haematoxylin and Eosin), picrosirius red, and an immunohistochemical test of HIF-1 Alpha.

First, it was possible to observe the difference in the portion where myocardial infarction was induced compared to general tissues through TTC staining, and as shown in a of FIG. 4, as the staining around the wall of the left ventricle was different, it was confirmed that macroscopic necrosis occurred in the corresponding area. Specifically, the necrosis rate (%) was measured and averaged by calculating the volume of the TTC-stained myocardial infarction site compared to the total left ventricle volume of 5 myocardial infarction models prepared in Example 1, and as a result, it could be confirmed that macroscopic necrosis with a necrosis rate of about 33(%) or more occurred (Table 4).

TABLE 4

| Sample NO. | Necrosis rate (%) |
|---|---|
| 1 | 26.54 |
| 2 | 24.03 |
| 3 | 27.14 |
| 4 | 54.91 |
| 5 | 31.60 |
| Average | 32.84 ± 11.30 |

In addition, the result of performing H&E staining at the initial stage and in 6 weeks after implantation of hollow construct, respectively, was shown in b of FIG. 4 (initial stage of implantation) and e of FIG. 4 (in 6 weeks after implantation). As shown in e of FIG. 4, an abnormal morphological change of cardiomyocytes compared to normal tissues could be observed microscopically.

Furthermore, the result of performing picrosirius red staining at the initial stage and in 6 weeks after implantation of hollow construct, respectively, was shown in c of FIG. 4 (initial stage of implantation) and f of FIG. 4 (in 6 weeks after implantation). As shown in f of FIG. 4, accumulation of collagen inside the necrotic myocardial tissue could be observed through the picrosirius red staining, and loss of function of general myocardial tissues due to myocardial infarction induction could be confirmed.

Finally, the result of performing immunohistochemical staining at the initial stage and in 6 weeks after implantation of hollow construct, respectively, was shown in d of FIG. 4 (initial stage of implantation) and g of FIG. 4 (in 6 weeks after implantation). As shown in g of FIG. 4, expression of HIF-1 alpha after implantation of hollow construct was clearly observed, and therefore, it could be confirmed that hypoxia occurred due to myocardial infarction.

### Example 4: Survival Rate Measurement

In order to confirm how successfully the method for producing an animal model with myocardial infarction according to one embodiment of the present invention produces an animal model with myocardial infarction, by a conventional method for producing an animal model with myocardial infarction as a comparative example, and the method for producing an animal model with myocardial infarction according to one embodiment of the present invention, animal models of 7 individuals, respectively, were prepared, and the survival rate after 5 weeks was measured and shown in Table 5. The myocardial infarction model prepared by the conventional method was prepared by a method of temporarily blocking blood flow inside a carotid artery using a generally used balloon catheter (Comparative example 1).

TABLE 5

| | Survival rate |
|---|---|
| Classification | after 5 weeks |
| Example 2 | 90% |
| Comparative Example 1 | 50% |

As shown in Table 5, the method for production according to one embodiment of the present invention showed a significantly high survival rate, and there was a problem in that different from mice for experiments, the pigs had a characteristic of a high degree of deviation of the myocardial structure and function, and the trend of numerical values for the blood flow and inner diameter of the coronary artery of each porcine individual was various, so it was difficult to perform a stable animal experiment, but the present invention is a model for studying myocardial infarction by customizing the diameter and through-hole of a hollow construct for inducing myocardial infarction in a unit of several tens of micrometers, and could stably secure various data and could control quantitative numerical values of the data, and could place on not only left anterior descending arteries but also inside other blood vessels, and thus, it could successively imitate myocardial infarction.

### Example 5: Change in Necrosis Rate and Ejection Rate Depending on Through-Hole Size of Hollow Construct

By comparing necrosis rate and ejection fraction values after producing hollow constructs having various through-hole sizes and implanting them, an effect of the through-hole size on inducing myocardial infarction was investigated. Specifically, after producing a hollow construct having a through-hole in a size of 24% of the hollow construct diameter (Example 5-1) and a hollow construct having a through-hole in a size of 14% of the hollow construct diameter (Example 5-2), they were implanted to 3-month-old Yorkshire×Landrace F1 crossbred castrated male pigs, respectively. In 28 days after implantation, by the substantially same method as (3) of Example 3, the necrosis rate and ejection fraction were measured and shown in Table 6.

TABLE 6

| Classification | Necrosis rate (%) | Ejection fraction (%) |
|---|---|---|
| Example 5-1 | 20 | 51 |
| Example 5-2 | 30 | 39 |

As shown in Table 6, as the size of the through-hole was relatively small, a more severe ejection fraction was measured due to an increase in myocardial blood pressure received from the inside of the left anterior descending coronary artery, and an increase in myocardial necrosis caused thereby could be observed.

1-8. (canceled)

9. A method for inducing myocardial infarction of an animal, comprising
preparing a cylindrical hollow construct with a through-hole extended in a longitudinal direction, which has an outer diameter ranging 70% to 130% of an inner diameter of a target blood vessel and an inner diameter with a certain size allowing blood flow to pass through, by 3-dimensional printing of polymer melt; and
inducing myocardial infarction by implanting the hollow construct inside the target blood vessel to partially block blood flow,
wherein the target blood vessel is a blood vessel of an animal except for human.

10. The method for inducing myocardial infarction according to claim 9, wherein the target blood vessel is one or more selected from the group consisting of a left anterior descending artery, a right coronary artery and a left circumflex artery.

11. The method for inducing myocardial infarction according to claim 9, wherein the preparing a cylindrical hollow construct is preparing so that an inner diameter of the cylindrical hollow construct is 50% or less of the outer diameter of the cylindrical hollow construct.

12. The method for inducing myocardial infarction according to claim 9, wherein the preparing a cylindrical hollow construct is preparing a length of the cylindrical hollow construct to a length of 1.5 to 5 mm.

13. The method for inducing myocardial infarction according to claim 9, wherein the polymer is one or more selected from the group consisting of polycaprolactone (PCL), poly(lactate-co-glycolate) (PLGA), poly lactic acid (PLA), polyurethane (PU), poly(lactide-co-caprolactone) (PLCL), polydioxanone (PDO), polystyrene (PS), poly(ethylene glycol) (PEG), poly(vinyl acetate) (PVA), polypropylene glycol (PPG), polyacrylamide (PAAm), polyglycolic acid (PGA), polymethylmethacrylate (PMMA), polyhydroxybutyrate (PHB), and polyvinylpyrrolidone (PVP).

14. The method for inducing myocardial infarction according to claim 9, wherein the implanting is injecting the cylindrical hollow construct into a carotid artery to implant it into a left anterior descending artery.

15. The method for inducing myocardial infarction according to claim 9, wherein the method for inducing myocardial infarction does not comprise removing the cylindrical hollow construct.

16. The method for inducing myocardial infarction according to claim 9, wherein the method for inducing myocardial infarction induces myocardial infarction while maintaining the cylindrical hollow construct without removing it.

17. The method for inducing myocardial infarction according to claim 9, further comprising breeding an animal in which the cylindrical hollow construct is implanted.

18. The method for inducing myocardial infarction according to claim 9, wherein the survival rate of the animal in 5 weeks after implanting the cylindrical hollow construct is 70% or more.

19. The method for inducing myocardial infarction according to claim 9, wherein the cylindrical hollow construct is implanted to an animal to induce one or more of the following (1) to (6):
(1) reduction of a left anterior descending artery diameter
(2) reduction of ejection fraction
(3) necrosis of cardiac tissue
(4) abnormal morphological changes in cardiomyocytes
(5) collagen accumulation inside myocardial tissue
(6) induction of hypoxia of myocardial tissue

20. The method for inducing myocardial infarction according to claim 9, wherein the animal is one or more selected from the group consisting of pigs, monkeys, chimpanzees, sheep, goats, cows, horses, camels, dogs and cats.

21. An animal model with myocardial infarction except for human, comprising an implantable hollow construct for inducing myocardial infarction in a target blood vessel,
wherein the implantable hollow construct for inducing myocardial infarction is a cylindrical hollow construct with a through-hole extended in a longitudinal direction, which has an outer diameter ranging 70% to 130% of an inner diameter of the target blood vessel and an inner diameter with a certain size.

22. The animal model according to claim 21, wherein the animal is one or more selected from the group consisting of pigs, monkeys, chimpanzees, sheep, goats, cows, horses, camels, dogs and cats.

23. The animal model according to claim 21, wherein the target blood vessel is one or more selected from the group consisting of a left anterior descending artery, a right coronary artery and a left circumflex artery.

24. The animal model according to claim 21, wherein the survival rate of the animal in 5 weeks after implanting the cylindrical hollow construct is 70% or more.

25. The animal model according to claim 21, wherein in the animal model, one or more of the following (1) to (6) are induced:
(1) reduction of a left anterior descending artery diameter
(2) reduction of ejection fraction
(3) necrosis of cardiac tissue
(4) abnormal morphological changes in cardiomyocytes
(5) collagen accumulation inside myocardial tissue
(6) induction of hypoxia of myocardial tissue

26. A method for preparation of an animal model with myocardial infarction except for human, comprising inducing myocardial infarction of the animal by implanting an implantable hollow construct for inducing myocardial infarction into inside of a target blood vessel of the animal,
wherein the implantable hollow construct for inducing myocardial infarction is a cylindrical hollow construct with a through-hole extended in a longitudinal direction, which has an outer diameter ranging 70% to 130% of an inner diameter of the target blood vessel and an inner diameter with a certain size to allow blood flow to pass through.

27. A method of screening a substance for prevention or treatment of a myocardial infarction, comprising
administering a candidate substance for prevention or treatment of the myocardial infarction to the animal model with myocardial infarction according to claim 21; and
selecting the candidate substance as a substance for prevention or treatment of myocardial infarction, when myocardial infarction of the animal model is improved after administering the candidate substance.

28. The method of screening according to claim 27, wherein the improvement of myocardial infarction is due to one or more of the following (1) to (7):
(1) maintenance or increase of diameter of a left anterior descending artery
(2) maintenance or increase of ejection fraction
(3) alleviation or delay of necrosis of cardiac tissue
(4) alleviation or delay of abnormal morphological changes of cardiomyocytes
(5) alleviation or delay of collagen accumulation inside myocardial tissue
(6) alleviation or improvement of hypoxia of myocardial tissue
(7) increase in survival rate compared to a control group not administered with the candidate substance.
